(19) United States
(12) Patent Application Publication
(10) Pub. No.: US 2013/0110110 A1
(43) Pub. Date: May 2, 2013

(54) POLYAXIAL EXTERNAL FIXATOR AND METHOD OF USING THE SAME

(71) Applicant: Gabriel Waisman, Tel-Aviv (IL)

(72) Inventor: Gabriel Waisman, Tel-Aviv (IL)

(21) Appl. No.: 13/807,983

(22) PCT Filed: Jul. 7, 2011
(86) PCT No.: PCT/IL11/00539

§ 371 Date: Jan. 2, 2013

Related U.S. Application Data
(60) Provisional application No. 61/361,963, filed on Jul. 7, 2010.

(51) Int. Cl. A61B 17/64

(52) U.S. Cl. CPC A61B 17/6458
USPC 606/59

(57) ABSTRACT

A flexible, light-weight, simple, modular-design Polyaxial External Fixator (PEF) for fixating bones is strong enough to hold the bones in their intended position. The fixator uses two or more preloaded pins or Kirschner-wires or non-preloaded screws. The PEF uses non-linear Kirschner-wires anchorage using external loading cages mounted in each arm of a polyaxial hinged external fixator. The hinges and the cages offer a high degree of configuration flexibility and modularity. The PEF may be provided in a kit of interchangeable parts. Most of the PEF parts are typically made of non-metallic radiolucent materials or made of any kind of metallic materials. Insertion and removal of Kirschner-wires is safe, fast and simple compare to screws.

### FIELD OF THE INVENTION

The present invention relates to skeletal bone fixation. More particularly, the present invention relates to a modular polyaxial bone fixator.

### BACKGROUND OF THE INVENTION

Fixators are being used for fixed location of two or more bone portions after fracture or after surgery with the aim to ensure healing and joining of the fractured or osteotomized bone portions or joints in their correct anatomical position. Articulated fixators become necessary for fixation of two or more fixator arms to the fracture fragment of the fracture bones to be treated or for optimal anatomical reduction. These fixation devices are usually mounted close to the bone through the skin and other soft tissues and are subsequently removed later, after optimal bone healing. However, where small bones of the hand, foot, face and skull, which are only thinly covered by skin and soft tissues, external fixators are the first choice to be used. The fixators maintain the bone parts in their correct position by pins, screws, or wires located in drilled bores in the bone, while extending outwardly through skin and other soft tissues.

In order to allow the patient to move his limb, it is important that the device is made light and small as possible, whilst being strong enough to support the bone portions against internal and external forces. On the other hand, it must permit angular and spatial alignment suitable with the anatomical reduction of the joint and the shape and size of the bones involved.

Fixators are being used for positioning two or more bone portions which have been displaced, often as a result of a fracture. They are often used in the course of or after surgical procedures such as reduction, realignment and correction. Fixators aim to ensure healing and joining of fractured bone portions or joints in their intended anatomical positions.

Articulated fixation devices are necessary for positioning two or more fixator arms upon bone fracture fragments to be treated for optimal reduction. These devices are usually mounted close to the bone through the skin and other soft tissue. The devices are removed after bone healing, thereby causing minimal inconvenience to the patient.

When the fracture is in the small bones of the hand, foot, face and skull, having only a thin cover of soft tissue and skin, external fixators are a common choice. Pins, screws, or wires are inserted into drilled bores in the bone and maintain the bone parts in their correct position, and extend outwardly through the skin.

It is a challenge to enable the patient to effectively use the body part installed with the fixator. Thus, external parts of the fixator should be made as light and as small as possible, whilst being strong enough to support the bone portions against internal and external forces. They must also permit angular and spatial alignment with the articulation of the joint and the shape and size of the bones involved.

U.S. Pat. No. 6,162,223 titled “Dynamic Wrist Fixation Apparatus for Early Joint Motion in Distal Radius Fractures” to Orsak et al.), discusses articulated fixators adapted to the natural movement of joints such as knees and wrists. The fixator includes two rods joined by a spring element. The spring element often comprises a removable catch used for restraining the spring's movement range before bone and joint reduction. The rods are used for fixating clamps inserted into bones located above and below a joint or a fractured bone. The clamps can be moved along the rods, to enable joint deflection and bringing of bone parts closer together.

US Patent application number 2006/0235383 titled “External Fixator” to Hollawell describes a fixator for serious, chronic and traumatic injuries to hands and feet. The system comprises a set of clamps, enabling the insertion of screws into bones and creating a multi-plane fixator.

US Patent application number 2009/0287212 titled “External Fixator” to Hirata et al. discusses an external fixator for small bones having ball-shaped connectors which enable relative movement of screws intended to be inserted into bone parts.

U.S. Pat. No. 5,941,877 titled “Hand Extended Fixator and Joint Mobilization and Distraction Device” to Viegas et al. describes a hand-fracture fixator enabling long term force exertion. This fixator is particularly relevant to burn-type trauma.

Prior art can be classified into three main classes:
Bi-dimensional fixators enabling movement on a single axis, such as described in U.S. Pat. No. 4,604,997, U.S. Pat. No. 5,653,707, U.S. Pat. No. 6,171,309, U.S. Pat. No. 5,683,389, U.S. Pat. No. 6,056,748, U.S. Pat. No. 5,662,649, and U.S. Pat. No. 5,709,681.
Tri-dimensional fixators enabling movement on more than one axis, such as described in U.S. Pat. No. 5,951,556, U.S. Pat. No. 5,941,877, U.S. Pat. No. 5,843,081, U.S. Pat. No. 3,036,691, and U.S. Pat. No. 5,429,637. These are mainly used on joints having a ball-shaped structure resembling a ‘pestle and mortar’. The external fixators assists the user in moving internal joints.
Rotational fixators such as U.S. Pat. No. 5,662,649, U.S. Pat. No. 5,803,924, U.S. Pat. No. 5,658,283 and U.S. Pat. No. 5,843,081. Such fixators typically comprise two interconnected elements acting as clamps and enabling relative bending-movement of bone portions.

### SUMMARY OF THE INVENTION

It is an object of the present invention to provide an external fixator of different sizes for fixating bones of any size before, during or after reduction, realignment and correction surgery. The fixator should be of light weight, simple design, but strong enough to hold the bones in their intended position.

It is another object of the present invention to provide an external fixator of modular design suitable for fixation fracture fragments of one or more bones, at least two adjoining bones, joints, or fragments at any angular alignment or any position whether the respective bones are long, short, or any other configuration.

It is another object of the present invention to provide an external fixator of modular design-suitable for fixation using K-wires, pins or screws.

The Polyaxial External Fixator (PEF) according to an exemplary embodiment of the invention is based on a unique bio-mechanical concept in cases using smooth K-wires or pins. At least one, pair (or optionally more) preloaded K-Wires or pins are used for polyaxial external fixation. In selected cases, PEF uses screws or threaded K-Wires.

Introduced in 1909 by Martin Kirschner, Kirschner wires (KW) are sterilized, sharpened, smooth stainless steel pins widely used today in orthopedic surgery. K-wires are manufactured in different sizes and are used so far to intraosseous, interfragmentary or/and intramedullary insertion in order to hold bone fragments together. The K-wires and pins are often driven into the bone through the skin (percutaneous pin fixation drilling) using a power or hand drill and commonly through a stab puncture or open skin incision for open reduction and fixation of broken or osteotomized bones.

In some embodiments, PEF introduces an innovative method for fixation in small bone fractures but may also be suitable for every size or type of bones. As can be seen in the illustration bellow, the novelty of the PEF external fixation is in the use of a basic unit of a Pair of Preloaded K-wires or pins and a non-linear KW anchorage concept, both fixed into an external loading lateral cage mounted interlocked in each arm rod of a polyaxial hinged external fixator.

PEF introduces the novel concept of intraosseous anchoring aof two or more smooth surface K-wires (basically smooth pins) into a holding-gripping fixation by applying two concepts:

1) Preloaded bone anchoring concept: preloading at least a pair of K-wires or pins after drilling into the bone and then approximation or separation of distal ends of the K-wires or pins, in order to tighten and gripping the proximal ends of the k-wires into the bone in a different direction of the original drill penetration and different pull out vector of the Kirshner Wire. By doing so, the invention intends to introduce a new alternative way for anchoring and griping bones before external fixation. Smooth K-Wires or pins may lower damage and tissue infection in comparison to screws.

2) Dual bone fixation concept: interlocking of internal fixation (intramedular or inter-fragmental K-wire or pin fixation) with an external fixation device.

According to some exemplary embodiments, PEF may perform one or few, or combination of thirteen different holding fixation possibilities based on the polyaxial device: 1) compression 2) distraction 3) abduction 4) adduction 5) flexion 6) extension 7) Clockwise motion 8) anti-clockwise motion 9) upper translation 10) lower translation 11) All of above combinations 12) Multiple transverse connections between two or more devices, and 13) Custom modular device elongation, unilateral or bilateral frame construction.

Some embodiments of PEF according to the current invention may provide one, few, or combination of the following advantages:
1. Compatibility/Adaptability—the device and method may be compatible with and suitable for use in a plurality of types and sizes of bones. Since using power drilling, there is no need for screwing, virtually no fragment is too small for inserting a smooth K-wire and large range of angles can be pursued.
2. Non rigid fixation—the PEF method of external fixation is flexible and intuitive. There is no need for strict calculation of approaching angle for K-wire drilling under fluoroscopic vision. Micromovements between fragments after non rigid fixation, promote and accelerate bone healing.
3. Safety—contrary to bone screws, K-wires have polished smooth surface. As a result, the metal-bone interface area is much smaller than a threaded screw and so the potential soft tissue damage or infection is significantly reduced. The pre-loaded triangular construction of a pair or more K-wires interlocked into the lateral PEF cage, resulting in a secure grip bone fixation, thus avoiding post-operative accidental over-penetration or pulling out the K-wires after they are adjusted and fixed into the lateral cage.
4. Time saving—the operation time for fixation may be significantly reduced as there is no need for strict measurements and calculations. The surgical technique may be forgiving, allowing an improved degree of freedom for K-wire insertion.
5. Ease of removal—K-wires are just pulled out applying a gently rotation.
6. Uniformity—A PEF kit may be composed of a limited number of repeating elements so that several kits can be combined for the treatment, no redundancies and no loss of critical parts can occur.
7. Flexibility—Polyaxial movements of the connecting bar allows for optimal alignment and reduction, including the unique translation mode.
8. Cost Effectiveness—The PEF may be made of plastic polymeric materials, optionally disposable, for example: ULTEM, Polyetherimide (PEI), PEEK, Ceramic, Resin reinforced fibers (such as carbon fibers, glass fibers, or Kevlar fibers) or the likes, and small metal parts such as stainless steel bolts and optionally nuts stainless steel). Optionally may be made of metal or any other material.
9. Simple and Easy to Use—There are few basic parts that can be assembled in different “Lego” like formations.
10. Versatile—The PEF devices may be manufactured in various sizes for different bone sizes and fractures locations.
11. Simple Removal in an Outpatient Department, avoiding a second operation in contrast to cases where removal of internal fixation is necessary.
12. Radiolucency—Most of the PEF parts may be made of radiolucent material. This allows clearer viewing of the bones during fixation and follow-up imaging under X-ray or Computer Tomography.

According to an exemplary embodiment, a hinged, light-weight, simple, modular-design Polyaxial External Fixator (PEF) which is strong enough to hold the bones in their intended position. The fixator uses two or more preloaded pins or K-wires but may use different bone screws. The PEF uses concept is based in a non-linear K-wire anchorage using connected to a lateral external loading cages mounted in each rod of a polyaxial hinged external fixator. The polyaxial possibilities of the central cage offer high degree of configuration flexibility and modularity. The PEF may be provided in a kit of interchangeable parts. Most of the PEF parts may be non-metallic and/or radiolucent. Insertion and removal of KW is safe, fast and simple compared to insertion and removal of screws.

According to an exemplary embodiment of the invention, polyaxial external fixator for fractured or osteotomized bones is provided, the fixator comprises: at least a first connecting rod (22); at least one bone penetrating elements (12), adapted to pass through bone; and at least two lateral cages (14), each having at least one recess (16) through which said bone penetrating elements (12) are held and fixed, wherein said lateral cage (14) is capable of connecting said bone penetrating elements (12) to said at least first connecting rod (22).

In some embodiments, the fixator, further comprises: at least a second connecting rod (22), wherein each of said at least first and second rods comprises at least one sphere (24); and at least one central cage joint (50, 26, 400), capable of flexibly joining said first and second rods (22) by interfacing with said spheres (24), and further capable of fixing the spatial orientation of said rods.

In some embodiments at least one of said spheres (24) is at one end of said at least one connecting rod (22), and said at least one sphere is adapted to be received and maintained within said at least one central cage joint (50, 26, 400).

In some embodiments the fixator further comprises a connecting element (24b, 102, 199, 198), said connecting element comprising a sphere (24, 24b), and is adapted to be attached to a connecting rod (22).

In some embodiments said at least one sphere (22) has a rough surface.

In some embodiments said at least one central cage joint (50, 26, 400) comprises: two substantially parallel and connected plates (52 and 53, 28a and 28b; or 40a and 48b) and wherein each one of said plates is provided with at least two bores (30 or 54) so as to allow confinement of said two spheres (24).

In some embodiments the top plate (52) and bottom plate (53) of said at least one central cage joint (50) are connected by side joint (54); and said central cage joint (50) further comprises at least one screw (56) capable of pulling said top plate (52) and bottom plate (53) together, thus casing said spheres (24) to be fixed within said bores (54).

In some embodiments the top plate (52) comprises a tapped hole (59); and said central cage joint (50) further comprises a fixating (locking) screw (58), wherein tightening said fixating screw (58) into said tapped hole (59) causes the end of said fixating screw (58) to exert pressure on said spheres (24).

In some embodiments at least two bone penetrating elements (12) are K-wires.

In some embodiments at least one of said bone penetrating elements (12) is a pin.

In some embodiments at least one of said bone penetrating elements (12) is a screw.

In some embodiments at least one of said lateral cages (14) comprises: a first and a second recesses (16) through which said bone penetrating elements (12) are held and fixed; a first and a second K-wire fixing screws 1(8), wherein fixing said bone penetrating elements (12) within said first and second recesses (16) is accomplished by tightening said first and second K-wire fixing screws (18) respectively.

In some embodiments at least one of said lateral cages (14) further comprises a first and a second K-wire fixing nuts (118), wherein fixing said bone penetrating elements (12) within said first and second recesses (16) is accomplished by tightening said first and second K-wire fixing screws (18) in said first and second K-wire fixing nuts (118) respectively.

In some embodiments at least one of said lateral cages (14) further comprises: a first hole (20) and a second hole (120), drilled in the body of said lateral cages (14), and sized to fit said connecting rod (22), wherein said first hole (20) and said second hole (120) are substantially perpendicular to each other, and wherein said first hole (20) and second hole (120) intersect; a tapped hole (121) drilled in the body of said lateral cages (14), substantially perpendicular to said first hole (20) and said second hole (120), and intersecting said first hole (20) and second hole (120); and a screw (19), wherein tightening said screw (19) within said tapped hole (121) causes the body of said lateral cages (14) to be firmly fixed to said connecting rod (22) inserted in one of said first hole (20) and second hole (120).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a schematically simplified view of the polyaxial external fixator system according to an exemplary embodiment of the present invention.

FIG. 2A schematically depicts a side view connector for K-wires fixation, showing the K-wire fixing screws and K-wire fixing nuts according to an exemplary embodiment of the present invention.

FIG. 2B schematically depicts an isometric view of the connector for K-wires fixation according to an exemplary embodiment of the present invention.

FIG. 2C schematically depicts another isometric view of the connector for K-wires fixation according to an exemplary embodiment of the present invention.

FIG. 2D schematically depicts production drawings of the connector for

FIG. 2E schematically depicts a fixator using connectors for fixation of bone having a fracture according to an exemplary embodiment of the current invention.

FIG. 2F schematically depicts another view of fixator according to an exemplary embodiment of the current invention.

FIG. 3 schematically depicts two rods having spheres at their ends adjustably connected using adjustable joint as known in the art.

FIG. 4 schematically depicts upper view of a clamp for flexibly joining two rods 22 according to an exemplary embodiment of the current invention.

FIG. 5A schematically depicts a side isometric view of an adjustable joint flexibly joining two rods, according to an exemplary embodiment of the current invention.

FIG. 5B schematically depicts a side view of an adjustable joint according to an exemplary embodiment of the current invention.

FIG. 5C schematically depicts production drawings of an adjustable joint, showing some exemplary dimensions according to an exemplary embodiment of the present invention.

FIG. 6 schematically depicts an isometric view of rod having sphere at one end according to an exemplary embodiment of the current invention.

FIG. 7A schematically depicts isometric view of connector according to an exemplary embodiment of the current invention.

FIG. 7B schematically depicts another isometric view of connector 102 according to an exemplary embodiment of the current invention.

FIG. 7C schematically depicts production drawings of connector, showing some exemplary dimensions according to an exemplary embodiment of the current invention.

FIG. 7D schematically depicts cross sectional view of selective dual purpose connector, allowing a choice of connecting sphere to the distal end of rod, or anywhere along rod such that neck and sphere protrude in a direction normal to the rod, according to an exemplary embodiment of the current invention.

FIG. 7E schematically depicts cross sectional view of simultaneous dual purpose connector, allowing a choice of connecting sphere to the distal end of first rod, anywhere along second rod such that neck and sphere protrude in a direction normal to second rod, or both first and second rods and 22b at once, according to an exemplary embodiment of the current invention.

FIG. 7F schematically depicts an isometric view of simultaneous dual purpose connector 198, according to an exemplary embodiment of the current invention.

FIG. 8A schematically illustrates a side connecting element according to an exemplary embodiment of the current invention.

FIG. 8B schematically depicts a snap off device for a side connecting element 24b according to an exemplary embodiment of the current invention.

FIG. 8C schematically depicts a side rod according to an exemplary embodiment of the current invention.

FIG. 8D schematically depicts a rod with: spherical thimble, a side rod, closed ring, side connecting element along it, according to an exemplary embodiment of the current invention.

FIG. 9A schematically illustrates an external fixator fixating fragments of a fractured distal radius in accordance with an exemplary embodiment of the present invention.

FIG. 9B schematically illustrates an external fixator fixating fractured finger phalanx in accordance with another exemplary embodiment of the present invention.

FIG. 9C schematically illustrates an external fixator applied to supracondylar humeral fracture in accordance with another exemplary embodiment of the present invention.

FIG. 9D schematically illustrates an external fixator applied to distal tibio-fibular fracture (Dual internal-external interlocking fixation) in accordance with another exemplary embodiment of the present invention.

FIG. 10 schematically illustrates an external fixator applied to a human foot for fixing an osteotomy (hallux valgus surgery) of first metatarsus deformity in accordance with another exemplary embodiment of the present invention.

### DETAILED DESCRIPTION OF THE INVENTION AND FIGURES

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The terms “comprises”, “comprising”, “includes”, “including”, and “having” together with their conjugates mean “including but not limited to”.

The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

In discussion of the various figures described herein below, like numbers refer to like parts. The drawings are generally not to scale. For clarity, non-essential elements were omitted from some of the drawing.

The present invention provides a unique and novel surgical device for universal bone fixation. The device is modular and is used for alignment, fixation, and stabilization of bones as well as other skeletal and health conditions.

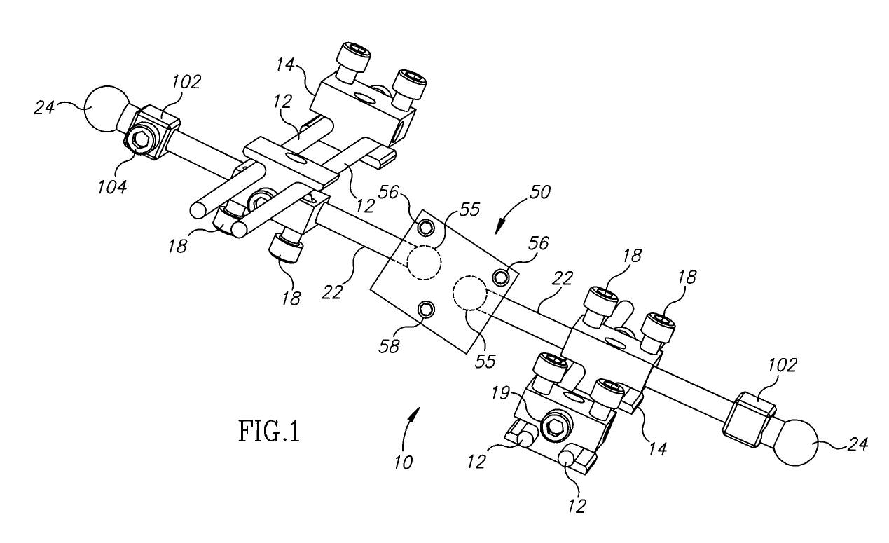

FIG. 1 illustrates the polyaxial external fixator system 10 according to an exemplary embodiment of the present invention.

Polyaxial external fixator 10 comprises bone penetrating elements 12 such as K-wires, pins or screws, optionally organized in pairs. In some embodiments, K-wire ends comprise threaded ends. It should be noted that K-wires or pins can be used in order to implement the fixator of the present invention and therefore in any case K-wires are mentioned, it may refer also to pins, screws, Steinman pins, Shantz pins, or other types of elongated devices that may be anchored to a bone. The K-wires commercially used are relatively flexible and have small diameter since they are adapted to be inserted through the bones that have to be fixed. Due to the flexibility and the diameter of the wires or pins, they can be inserted through parts of the body such as fingers and used with a plurality of types of fractures of the bone. K-wires 12 are fixed to a lateral cage 14 which is attached to a connecting rod 22. In the depicted embodiment, lateral cage 14 is provided with two opposite recesses 16 through which K-wires 12 pass and fixed. In some embodiments, the user applies mild approximation or separation of both distal ends of a flexible pair of k-wires, in one side or both sides of the fixated bone (pre-loading), before the k-wires are introduced to recesses 16 of lateral cage 14. This preloading can prevent accidental over-penetration, out-sliding or dislodging of the smooth k-wires from fixated bones. Preloading is not required or recommended when using bone screws. It should be noted that K-wires 12 are available in a variety of thicknesses. An adequately sized recess 16 may accommodate a range of K-wires diameters. Optionally a plurality of lateral cages 14 may be made having a range of sizes of recesses 16 to accommodate different diameters of K-wires.

It should be noted that the smoothness, thinness and flexibility of the K-wires renders the fixator 10 of the present invention characteristics of a minimal invasive device which can be inserted through the skin and the fractured bones by drilling, optionally without the need to open the tissues when this is not necessary. The fixator 10 can also be used in open surgeries.

Other parts and elements marked in this figure such as adjustable joint 26, connecting the two connecting rods 22 will be detailed in the following figures.

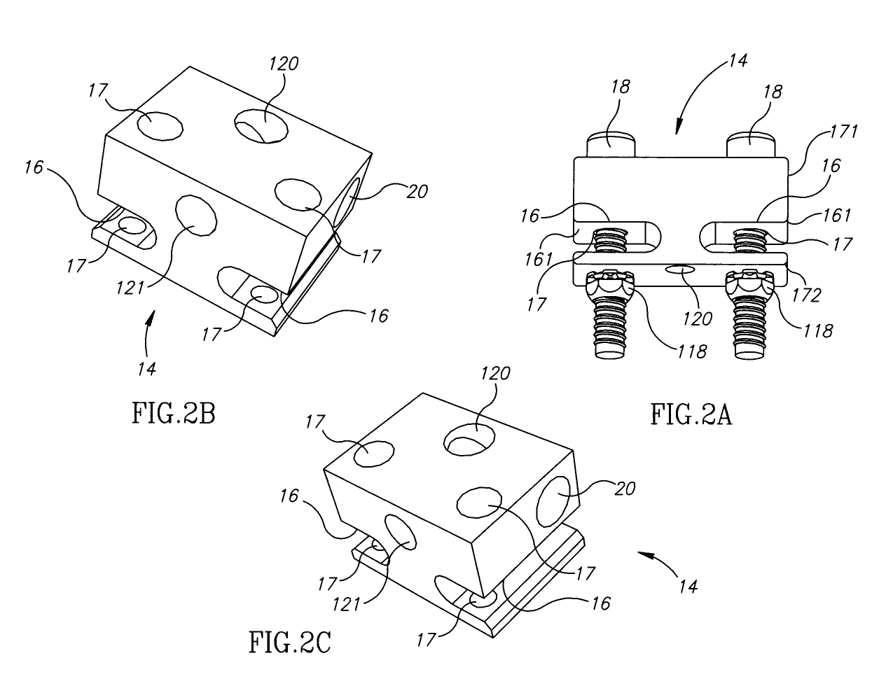

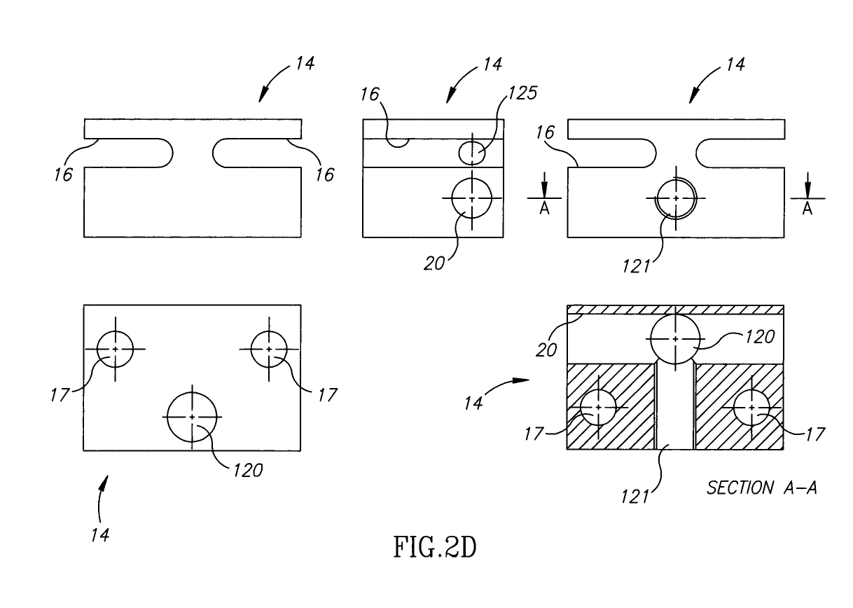

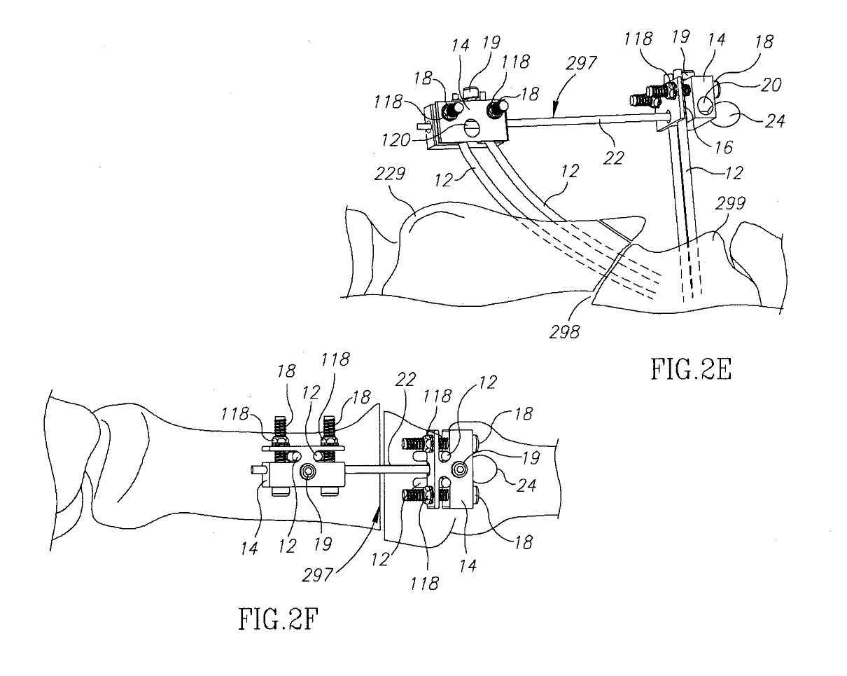

Reference is now made to FIGS. 2A 2D, illustrating lateral cages 14 for K-wires fixation and their applications.

FIG. 2A schematically depicts a side view lateral cage for K-wires fixation, showing the K-wire fixing screws 18 and K-wire fixing nuts 118 according to an exemplary embodiment of the present invention.

FIG. 2B schematically depicts an isometric view of the lateral cage for K-wires fixation according to an exemplary embodiment of the present invention.

FIG. 2C schematically depicts another isometric view of the lateral cage for K-wires fixation according to an exemplary embodiment of the present invention.

FIG. 2D schematically depicts production drawings of the lateral cage for K-wires fixation, showing exemplary dimensions according to an exemplary embodiment of the present invention.

Lateral cage 14 may be a rectangular or cubic-like member having two opposite recesses 16 adapted to receive K-wires or pins (the K-wires are not shown in this figure) that pass through the recesses.

Preferably, K-wires are not inserted through a hole in the lateral cage as in prior art devices that limit the use of the fixators. Since the K-wires pass through an open recess 16, they can be introduced straight from the open side 161 of recess 16. This feature allows flexibility, “free hands adjustability” and simplicity to the sequence of the procedure of fixing the bones. In order to fix the K-wires within lateral cages 14 in a certain position, holes 17 are provided in lateral cage 14 and allowing screws 18 (for example Allen screws) to be used with nuts 118 and firmly hold the K-wires by compressing recess 16. Optionally, holes 17 are tapped within the thick lip 171 of lateral cage 14, and untapped within the thin lip 172 lateral cage 14, such that a screw may be inserted through the untapped hole and tighten to the tapped part of the hole in the thick lip 171 of lateral cage 14, thus compressing recess 16.

It should be mentioned that optionally lateral cage 14 may be provided with at least one hole (for example K-wire hole 125 seen in FIG. 2D), or a plurality of holes through which rods or K-wires can pass as needed. One can design a series of lateral cages through which holes pass in different directions in order to further increase the modularity of the fixator.

To secure lateral cage 14 onto modular arm 22, modular arm 22 is inserted in one of holes 20 or 120 and a screw 19 is tightened into tapped hole 121 firmly holding lateral cage 14 onto modular arm 22.

FIG. 2E schematically depicts a fixator 297 using lateral cages 14 for fixation of bone 299 having a fracture 298 according to an exemplary embodiment of the current invention. It should be noted that rod (arm) 22 is typically made with a diameter larger than that of a neighboring K-wire, to supply more stability to the structure constructed by the cages.

FIG. 2F schematically depicts another view of modular arm 297 according to an exemplary embodiment of the current invention.

Reference may also be made to FIG. 1. FIG. 1 show Allen screws 18 that are provided in the holes 17 and used with nuts 118 to compress recesses 16 so as to maintain and hold K-wires 12 that are within the recess 16 of the lateral cage 14.

Lateral cages 14 are provided with additional holes 20 and 120 through one of which a connecting rod 22 can be inserted in one of two substantially orthogonal/perpendicular directions respectively.

Connecting rods 22 are provided to fixator 10 or 297 and are sized to be adapted for insertion through holes 20 or hole 120 in lateral cage 14. The positioning of the rods within the hole of lateral cage 14 is typically maintained using screws 19 (for example an Allen screw). Optionally screws 19 are similar or identical to screws 18 that are used to fixate the K-wires. Alternatively screws 19 are of different size.

For the fixator 297, which used a single modular arm 22, any straight or even a bent rod of appropriate diameter may be used. Modular arm 22 may also be referred to herein as “connecting rod” or just “rod”.

However, in some embodiments modular arm 22 comprises or is connectable to a small sphere 24 at its end. Two modular arms 22, having spheres at their ends can be connected to each other through spheres 24 using an adjustable joint such as 26, 400 or 50 as will be shown below.

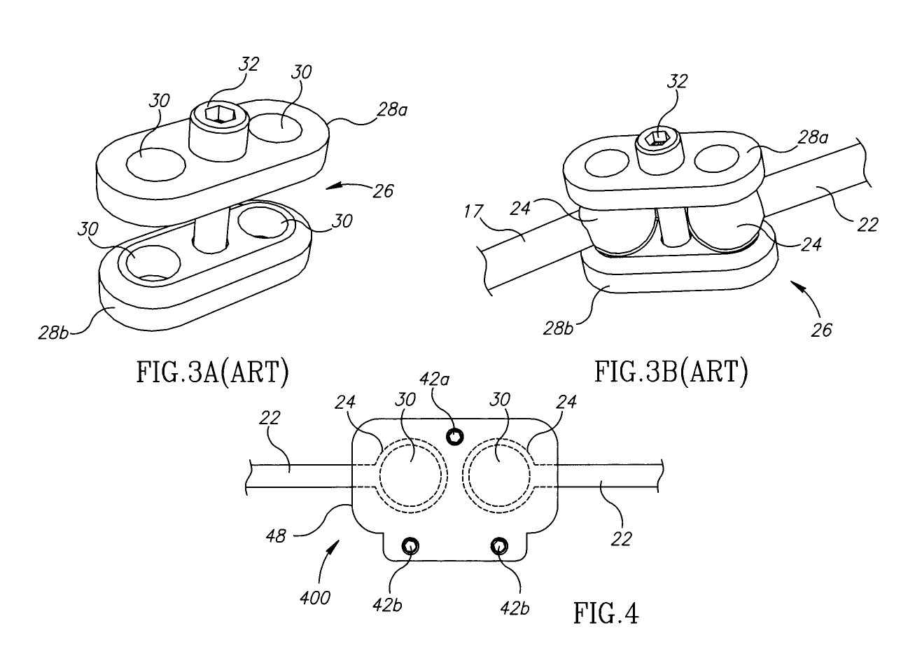

FIG. 3 schematically depicts central cage 26 serving as an adjustable joint for flexibly joining two modular arms 22.

Central cage 26 is shown in an isometric view in FIG. 3. The figure schematically depicts two modular arms 22 having spheres 24 at their ends adjustably connected using central cage 26 according to an exemplary embodiment of the current invention.

Central cage 26 is preferably designed to receive both ends of the connecting rods coming from two connectors. Two opposing plates 28a and 28b are provided wherein each plate is provided with at least two bores 30 each sized to fit sphere 24. A connecting screw 32 enters a hole in top plate 28a and engages tapped hole in bottom plate 28b so as to draw the plates 28a and 28b one towards the other.

Alternatively or additionally a nut (not seen in this figure) is used tor tightening or locking connecting screw 32. Bores 30 in plates 28 may be through holes or indentations in the faces of the plates facing each other. Bores or indentations 30 are organized so that two bores from the two plates can be placed opposite one another. As can be seen in FIG. 3B, each one of modular arms/rods 22 may be rotated around its long axis as well as swiveled (in the plane of the plate) and to some degree tilted (normal to the plate)in respect to the central cage 26 within the sockets formed by the pair of bores 30, as long as connecting screw 32 is only loosely tightened. However, once connecting screw 32 is firmly tightened, the orientation of the two rods respect to the central cage 26 and thus respect to each other is fixed. Facets 399 on plates 28a and 28b increases the range in which rods 22 can be tilted. Using central cage 26, modular arms 22 may be set at an angle to each other or even parallels but shifted in respect to each other.

FIG. 4 schematically depicts upper view of a clamp 400 for flexibly joining two modular arms 22 according to an exemplary embodiment of the current invention.

Clamp 400 is similar to central cage 26 except that clamp 400 uses three screws 42a, 42b and 42c instead of a single screw 32 of central cage 26. Consequently, clamp 400 may provide a more secured fixation of modular arms 22. Additionally, clamp 400 may be made of X-ray transparent/radiolucent (non-metallic) material. In some embodiments, screws 42 are used with nuts, in other embodiments, one of the plates 48 (only the top plate 48a is seen in this top view) has tapped holes accepting screws 42.

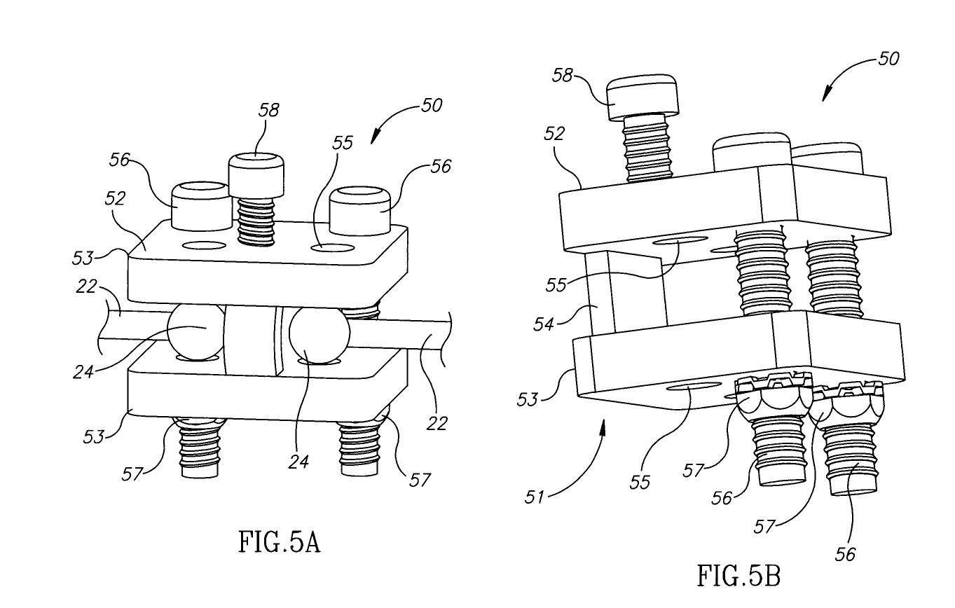

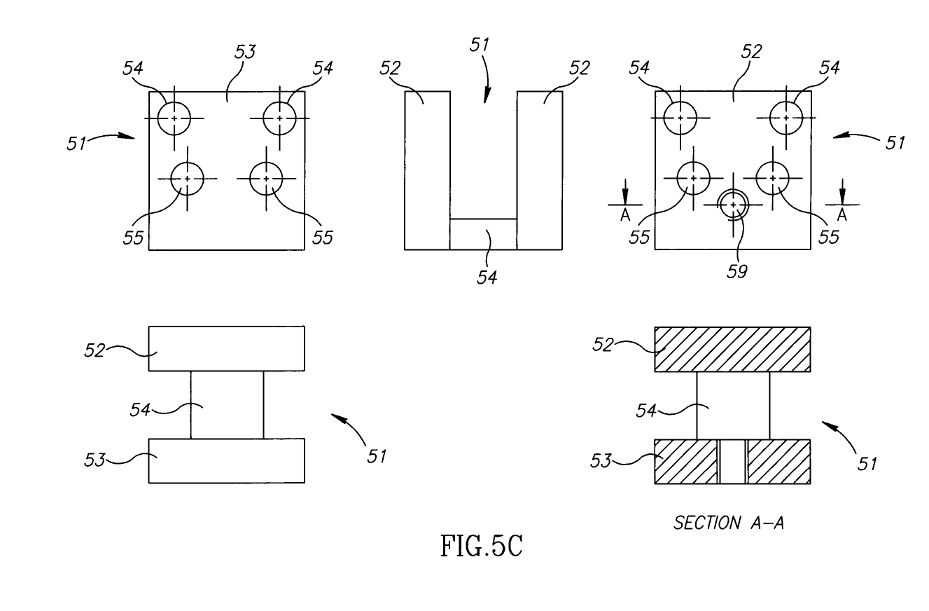

FIGS. 5A to 5C schematically depict an adjustable joint/central cage 50 for flexibly joining two rods 22 according to an exemplary embodiment of the current invention.

FIG. 5A schematically depicts a side isometric view of an adjustable joint 50 flexibly joining two rods 22 according to an exemplary embodiment of the current invention.

FIG. 5B schematically depicts a side view of an adjustable joint 50 according to an exemplary embodiment of the current invention.

FIG. 5C schematically depicts production drawings of an adjustable joint 50, showing some exemplary dimensions according to an exemplary embodiment of the present invention.

Adjustable joint 50 comprises a U shaped body 51 (seen best in FIGS. 5C and 5D) having a top plate 52 and a bottom plate 53 connected by side joint 54. Top plate 52 and bottom plate 53 has bores or indentations 55 sized to accept spheres 24 at the ends of rods 22. Top plate 52 and bottom plate 53 has holes 54 for screws 56 that together with nuts 57 pushes the tow plates together (slightly bending side joint 54 and/or the plates). Optional locking fixating screw 58, screwed in tapped hole 59 (seen in FIG. 5E) in top plate 52 pushes spheres 24 at the ends of two rods 22 and provides additional securing of their orientation and final fixation relative to the adjustable joint 50 and thus relative to each other.

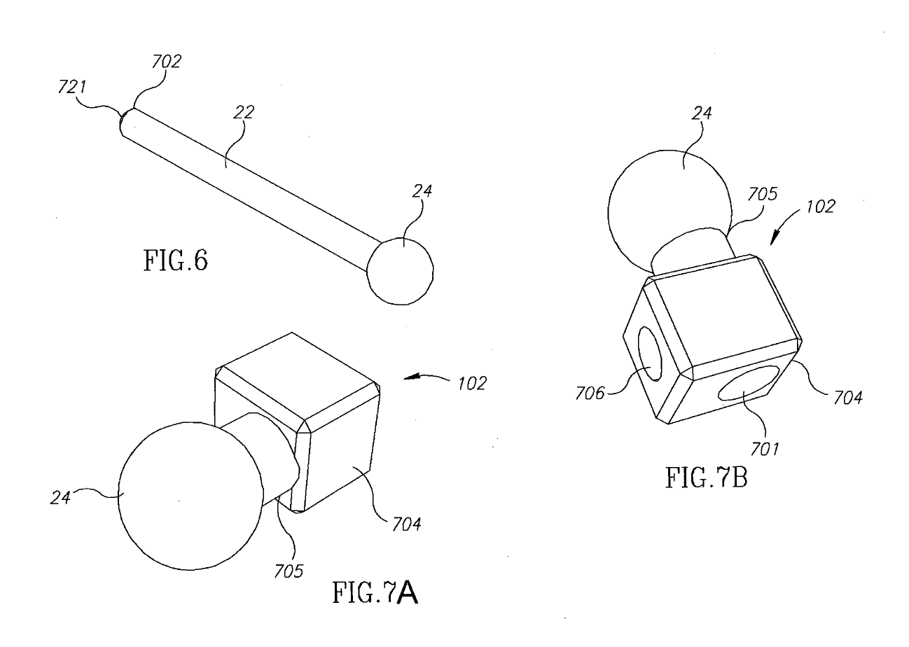

FIG. 6 schematically depicts an isometric view of rod 22 having sphere 24 at one end according to an exemplary embodiment of the current invention. In some embodiments, sphere 24 has a rugged surface, for example having engraved slits upon its surface. Optionally, the slits can have a diamond like pattern or other patterns which provides additional friction between the sphere and bores or indentation 30 or 55.

Rod 22 may be available at a plurality of standard lengths, and may be ordered or cut to desired length. End 721 of rod 22 may have a facet 702 to ease insertions into holes such as 20 or 120 in lateral cage 14, or other holes disclosed below.

Alternatively, rods without a sphere at the end, or rods with spheres at two ends may be available.

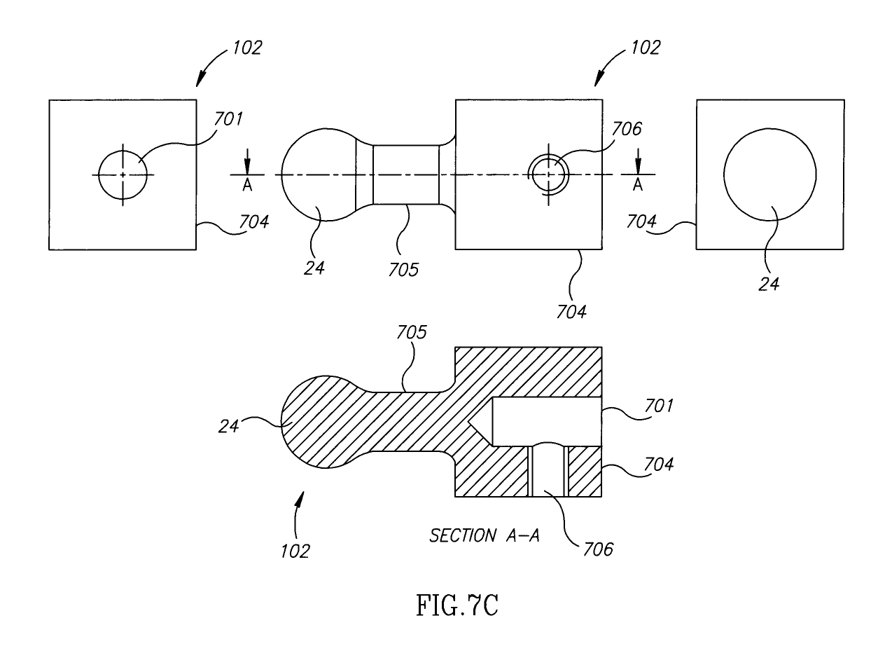

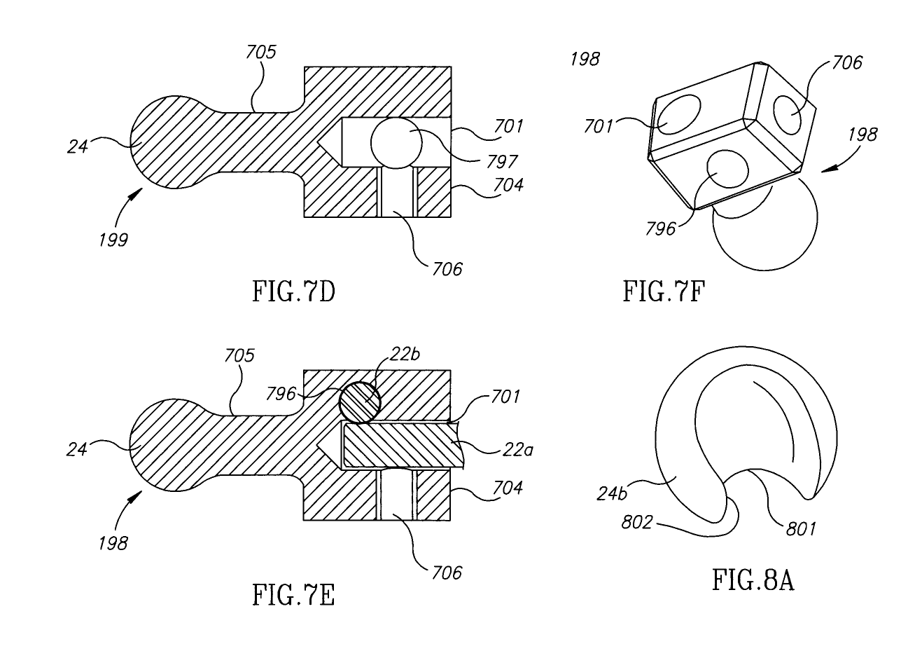

FIGS. 7A to 7C schematically depicts distal end unit 102 having a sphere 24 serving as a cap to be attached to end 721 of rod 22 according to an exemplary embodiment of the current invention.

FIG. 7A schematically depicts isometric view of distal end unit 102 according to an exemplary embodiment of the current invention.

FIG. 7B schematically depicts another isometric view of distal end unit 102 according to an exemplary embodiment of the current invention.

FIG. 7C schematically depicts production drawings of distal end unit 102, showing some exemplary dimensions according to an exemplary embodiment of the current invention.

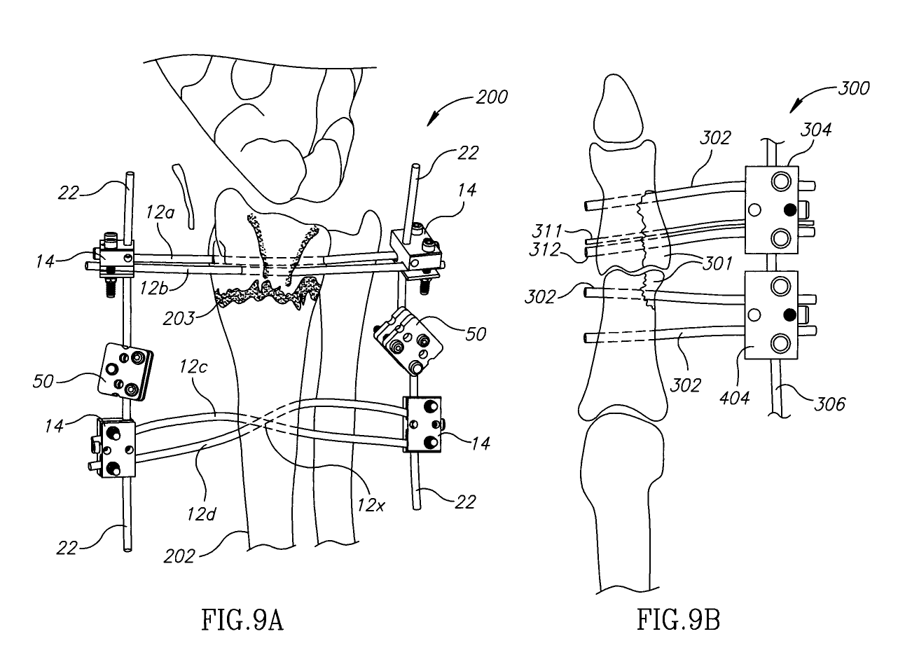

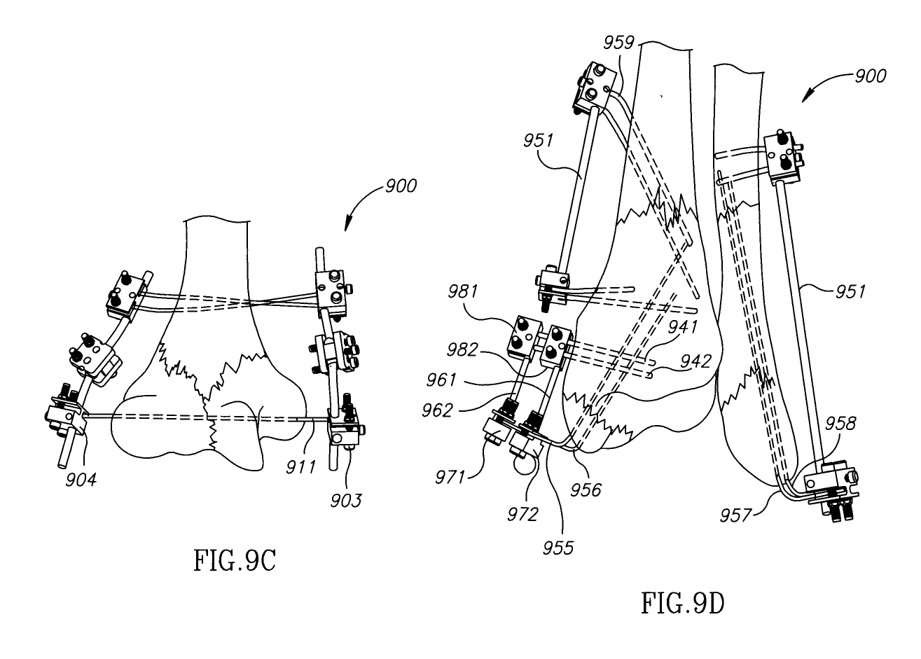

Distal end unit 102 comprises a body 704 having a hole 701 sized to fit the end 721 of rod 22, and a tapped hole 706 for connector fixation screw 104 (seen in FIG. 9B). A short neck 705 connects sphere 24 to body 704 of connector 102. By inserting the end 721 of rod 22 into hole 701 and tightening connector fixation screw 104 a second sphere 24 is added to the distal end 721 of the rod 22, creating a dual-sphere rod.

FIG. 7D schematically depicts cross sectional view of a selective dual purpose distal end unit 199, allowing a choice of connecting sphere 24 to the distal end 721 of rod 22, or anywhere along rod 22 such that neck 705 and sphere 24 protrude in a direction perpendicular to rod 22, according to an exemplary embodiment of the current invention.

In this exemplary embodiment, a through hole 797 is drilled in body 704 of connector 199. The user has a choice of inserting the distal end 721 of rod 22 via hole 701, or to insert rod 22 into hole 797 which is perpendicular to hole 701. Connector fixation screw 104 inserted and tightened into tapped hole 706 fasten rod 22 to selective dual purpose distal end unit 199.

FIG. 7E schematically depicts cross sectional view of simultaneous dual purpose distal end unit 198, allowing a choice of connecting sphere 24 to the distal end of first rod 22a, anywhere along second rod 22b such that neck 705 and sphere 24 protrude in a direction perpendicular to second rod 22b, or both first and second rods 22a and 22b at once, according to an exemplary embodiment of the current invention.

In this exemplary embodiment, a shifted through hole 796 is drilled in body 704 of distal end unit 198. The user has a choice of inserting only the distal end of a first rod 22a via hole 701, or to also insert a second rod 22b into shifted through hole 796 which is normal to hole 701 and shifted in respect to hole 701. Connector fixation screw 104 inserted and tightened into tapped hole 706 fasten first rod 22a to simultaneous dual purpose connector 198. If a second rod 22b is present in shifted hole 796, fixation screw 104 pushes on the first rod 22a in such that it pushes on second rod 22b and both first and second rods 22a and 22b become fixed to simultaneous dual purpose distal end unit 198.

FIG. 7F schematically depicts an isometric view of simultaneous dual purpose distal end unit 198, according to an exemplary embodiment of the current invention.

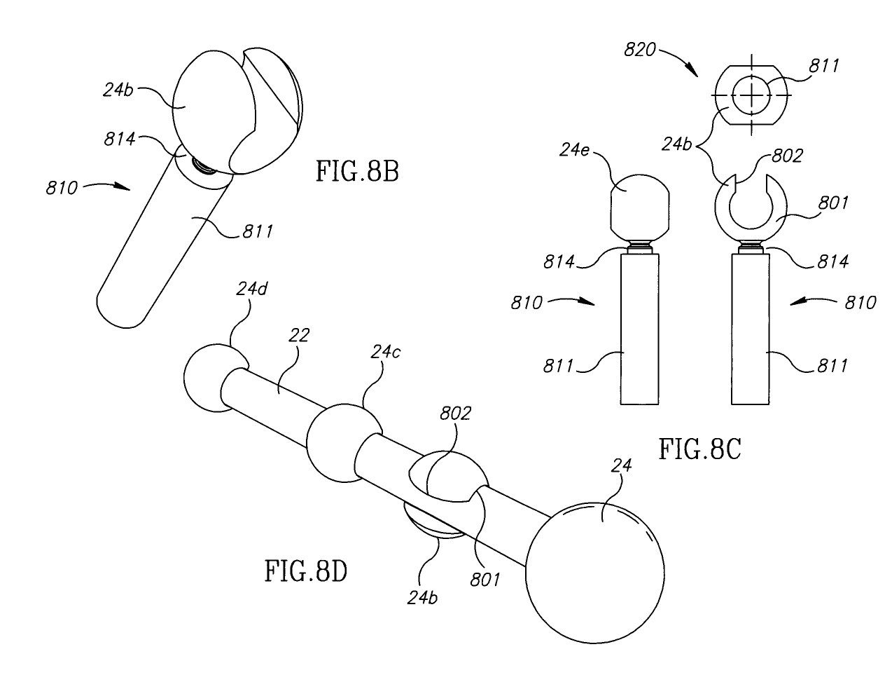

FIG. 8A schematically illustrates a “C-washer” 24b serving as a side connecting element according to an exemplary embodiment of the current invention.

C-washer 24b is shaped and sized similarly to sphere 24, but with a hole 801 sized to fit rod 22 in it. In the exemplary embodiment of FIG. 8A, hole 801 comprises a gap 802, making C-washer 24b a U shaped spherical open ring. Gap 802 allows insertion of rod 22 into the C-washer 24b through said gap, specifically when C-washer 24b is made of somewhat elastic material.

In some embodiments, the hole in C-washer has a smaller diameter, for example sized to fit one of the standard K-Wires 12. Using such C-washer side connecting element enables fixing and holding a K-wire to adjustable joint 26 or 50. Using adjustable joint for holding a K-wire enables greater flexibility in orientation of the K-Wire.

Although C-washer 24b is also referred to herein as “side connecting element”, it may be placed at the end of rod such as rod 22 and used in a similar manner to distal end unit 102.

In operation, C-washer 24a is inserted into an adjustable joint such as adjustable joint 26 or 50, and the forces exerted by the adjustable joint hold the C-washer in place relative to the rod 22 which is in it.

Alternatively, the hole is a through hole making it shaped as a closed ring 22c seen in FIG. 8D.

Alternatively, the hole is a partial hole making it shaped as a spherical thimble 22d to be inserted on a distal end of rod 22 seen in FIG. 8D.

FIG. 8B schematically depicts an isometric view of a snap off device 810 for connecting element 24b according to an exemplary embodiment of the current invention.

Snap off device 810 comprises a handle 811 connected with a thin neck 814 to the side element-washer 24b. After placing connecting element 24b on rod 22, handle 811 may be disconnected by breaking off, cutting or sawing thin neck 814. It is important that thin neck 814 is not overly thin, so it does not break while still being fitted upon connecting arm 22 during surgery.

FIG. 8C schematically depicts production drawings of a snap off device 810 according to an exemplary embodiment of the current invention.

Snap off device comprises a handle 811 connected to a perforated side connecting sphere 24b. Side connecting sphere 24e may be of similar in size and construction as elements 24b, 24c or 24d. In operation, connecting sphere 24b is inserted into an central cage such as central cage 26 or 50 or clamp 400, and the forces exerted by the adjustable joint hold the connecting element in place relative to the rod 22 which is in it.

FIG. 8D schematically depicts a rod 22 with: spherical thimble 22d, closed ring 22c, side connecting element 24b along it, according to an exemplary embodiment of the current invention.

Returning to FIG. 1; Small spheres 24 that are connected to rods 22 are maintained and confined between the two opposite bores of plates 52 and 53 without having the ability to move relative to the plates of the central cage 50 when the plates are pressed together by connecting screw 56. Central cage 50 serving as adjustable joint is preferably adapted to receive two small spheres 24; therefore, each joint is adapted to join at least two connecting rods.

It is important to notice that since the adjustable joint is holding at least one sphere that is connected to a rod, the rod can be maintained in any angle relative to the joint or the other rod that is oppositely connected. This renders the fixator features that are not possible in prior art fixators and especially translational fixation.

Connecting rods 22 can be connected to one another using the sphere that is connected at its end as shown herein but can be also be connected on its side to provide other connection configurations. Connecting element 24b has a rounded shape that is adapted to be enclosed on connecting rod 22. Connecting element 24b can be confined within joint 26 in the same manner shown herein before for sphere 24. This feature provides even a greater variety of new possibilities of enforcing the fixation of the present invention.

Small spheres 24 or connecting elements 24b are preferably made of a material having a rough surface adapted to prevent the spheres from unintentional movements within the joint after it is firmly held between the plates.

It should be emphasized that other types of adjustable joints can be employed by the embodiment of the present invention.

Reference is now made to FIGS. 9A to 9D illustrating uses of polyaxial fixators in according to another exemplary embodiment of the present invention.

The polyaxial external fixator demonstrates some of the flexible modularity of the fixator while using a plurality of the same connectors and adjustable joints that were shown herein before. Additional connecting rods or wires maybe used in the fixator in order to render strength to the fixator. In order to better understand the invention and comprehend the manner it can be used and its modularity, the modular fixator relative to the bones while fixating fractures is being demonstrated.

Reference is now made to FIG. 9A illustrating an external fixator 200 fixating fragments of a fractured distal radius in accordance with an exemplary embodiment of the present invention.

External fixator 200 fixates bone fragments of the radius 202 that are fractured 203. Two pairs of K-wires (first pair 12a and second pair 12b; and 12c and 12d) are inserted through the bones. The K-wires 12 are interconnected using lateral cages 14 on both sides. Lateral cages 14 are strengthen through connecting rods 22 that are joined using adjustable joints or central cages 50. This figure clearly shows the flexibility (non-rigid fixation) of the K-wires and the modularity (bilateral frame construction) of the fixator. In the depicted example, second pair of K-wires 12c and 12d are crossed in “X” configuration as seen by dashed lines showing the relative positions of the K-wires within the bones. By sliding and/or rotating the lateral cages 14 along rods 22, and by changing the relative positions of rods 22 using central cages 50, the bone fragments may be held in desired position relative to each other.

Forcing the k-wires (as seen in the case of 12c and 12d), the wires, which are smooth are firmly held to the bone. However, once the K-wires are freed from the lateral cages 14, they may be removed with ease.

Reference is now made to FIG. 9B illustrating an external fixator 300 fixating fractured finger phalanx in accordance with another preferred embodiment of the present invention.

This example (unilateral and non-articulated frame construction) clearly shows the simplicity and modularity capabilities of the fixator of the present invention so as to allow fixation of fractures in very small bones such as phalanxes (see two small diameter pins 311 and 312 fixating a small fracture fragment 301 passing and attached into the same side recess of lateral cage 304. Fixator 300 is a one-sided fixator. K-wires 302 are inserted through very small bones and very small fractures since the variability of the wire's diameter enables the physician to use very thin K-wires. Since one of the goals of the present invention is to enable the patient to continue his every-day duties with minimal disturbance to other fingers, only one arm of the fixator is held by lateral cages 304. It should be emphasized that preloading (angled drilled k-wires and further flexible approximation of both before attachment into lateral cage's 304 side recesses) can be performed in this one side case in order to prevent pull-out (withdrawal) or over-penetration of the K-wires from or into the bone and to enable the one-side connection to the connectors.

Fixation can be achieved for example and without limitation by inserting two K-wires into the lateral cage in parallel. Alternatively K-wires can be inserted such that they are in V-shaped or Y-shaped configuration. The wires are then pressed together within the lateral cage.

Another feature that can be observed in this example is the use of two K-wires having different diameters that are being held by the same lateral cage. The use of such different K-wires further establishes the modularity and versatility of the fixator of the present invention.

Connectors 304 are further strengthen and held by a connecting rod 306 (such as rod 22 for example) that passes through holes in both connectors 304.

It should be noted that it is optional to provide a fixator made of disposable materials in order to reduce the costs that of re-using and re-sterilizing such external fixator.

As shown herein before, there is a vast amount of possibilities to interconnect the K-wires between the connectors. It should be mentioned that the K-wires can also be transfixed in an X shape within the bone in order to increase the stability of the fixation.

FIG. 9C schematically illustrates an external fixator 900 applied to a supracondylar humeral fracture in accordance with another exemplary embodiment of the present invention.

In this embodiment, a single K-wire 911 is held at both ends by two lateral cages 903 and 904.

FIG. 9D schematically illustrates an external fixator 950 applied to distal Tibio-Fibular fracture (Dual internal-external interlocking fixation) in accordance with another exemplary embodiment of the present invention.

In this exemplary embodiment, K-wires such as 955, 956 957, 958 and 959 are bent. The dash lines schematically show the extent that these K-wires may penetrate the bones.

In this exemplary embodiment, a metal rod 961 is used for providing strength to the fixator.

In this exemplary embodiment, a second rod 962 is used in parallel rode 962 for providing strength and stability to the fixator. K-wires 955 and 956 are held to rods 961 and 962 using lateral cages 971 and 972 respectively. Similarly, lateral cages 981 and 982 hold K-wires 941 and 942 to rods 961 and 962 respectively.

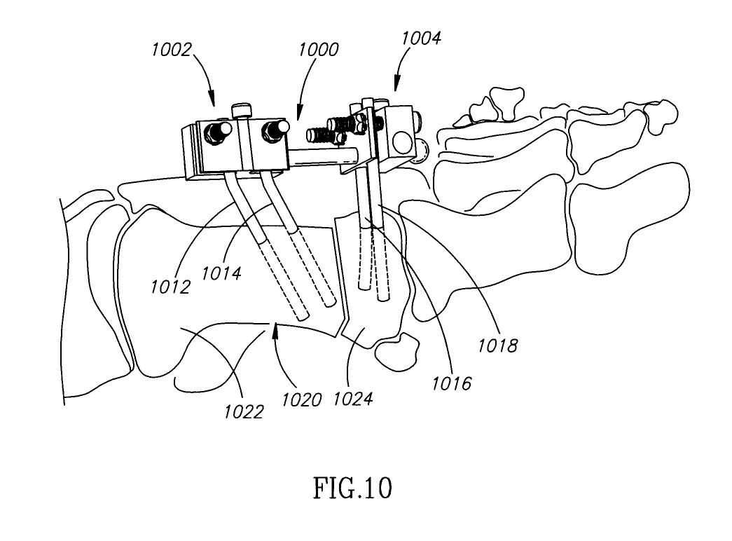

FIG. 10 schematically illustrates an external fixator applied to a human foot for fixing an osteotomy (hallux valgus surgery) of first metatarsus deformity in accordance with another exemplary embodiment of the present invention. A PEF embodiment 1000 is shown, having two lateral cages 1002 and 1004 connected by a rod (arm). Lateral cage 1002 is seen from a front view, and lateral cage 1004 is seen from a side view, as it is positioned substantially orthogonally to cage 1002.

In this view, K-wires 1012 and 1014 are seen one next to the other, fixated within lateral cage 1002; K-wires 1016 and 1018 are seen one behind the other, fixated within lateral cage 1004.

Incision 1020 can be made to separate two bone portions 1022 and 1024 from their previous deformity to a new position. After the bone incision 1020, the two portions can be fixed in their proper position relative to each other. K-wires 1012 and 1014 are seen inserted into bone portion 1022 and K-wires 1016 and 1018 are seen inserted into bone portion 1024.

The inventive PEF system and method may be used in a plurality of other surgical procedures, including in cardiovascular surgery, maxillofacial surgeries, trauma, orthopedic surgery such as but not limited to hand and foot surgeries, neurosurgery or the like. The invention may be used as temporary or final fixation during mass casualty events such as often happens in military or natural disasters. The invention may be useful in field hospitals. Veterinary surgery may also benefit from the present invention.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

It should be clear that the description of the embodiments and attached Figures set forth in this specification serves only for a better understanding of the invention, without limiting its scope as covered by the following Claims.

It should also be clear that a person skilled in the art, after reading the present specification can make adjustments or amendments to the attached Figures and above described embodiments that would still be covered by the following Claims.

1. A polyaxial external fixator for fractured or osteotomized bones comprising:
at least a first connecting rod;
at least two bone penetrating elements, adapted to pass through bone; and
at least two lateral cages, each having at least one recess through which said bone penetrating elements are held and fixed, wherein each of said lateral cages is capable of connecting said bone penetrating elements to said at least first connecting rod.

2. The fixator as claimed in claim 1, further comprising:
at least a second connecting rod, wherein each of said first rod and second rod comprises at least one sphere; and
at least one central cage joint capable of flexibly joining said first and second rods by interfacing with said spheres, and further capable of fixing the relative orientation of said rods.

3. The fixator as claimed in claim 2, wherein at least one of said spheres is at one end of said at least one connecting rod, and wherein said at least one sphere is adapted to be received and maintained within said at least one central cage joint.

4. The fixator as claimed in claim 2, further comprising a connecting element, said connecting element comprising a sphere, and configured to be attached to a connecting rod.

5. The fixator as claimed in claim 2, wherein said at least one sphere has a rough surface.

6. The fixator as claimed in claim 2, wherein said at least one central cage joint comprises two substantially parallel and connected plates, each one of said plates provided with at least two bores so as to allow confinement of said two spheres.

7. The fixator as claimed in 6, wherein:
said top plate and bottom plate of said at least one central cage joint are connected by side joint; and
said central cage joint further comprises at least one screw capable of pulling said top plate and bottom plate together, thus causing said spheres to be fixed within said bores.

8. The fixator as claimed in 7, wherein:
said top plate comprises a tapped hole (59); and
said central cage joint further comprises a fixating screw, wherein tightening said fixating screw into said tapped hole causes the end of said fixating screw to exert pressure on said spheres.

9. The fixator as claimed in claim 1, wherein at least one of said two bone penetrating elements is a Kirschner-wire.

10. The fixator as claimed in claim 1, wherein one of said at least two bone penetrating elements is a pin.

11. The fixator as claimed in claim 1, wherein at least one of said two hone penetrating elements is a screw.

12. The fixator as claimed in claim 1, wherein at least one of said lateral cages comprises:
a first recess and a second recess through which said bone penetrating elements are held and fixed;
a first Kirschner-wire fixing screw and a second Kirschner-wire fixing screw, wherein fixing said hone penetrating elements within said first and second recesses is accomplished by tightening said first and second Kirschner-wire fixing screws, respectively.

13. The fixator as claimed in claim 12, wherein at least one of said lateral cages further comprises a first Kirschner-wire fixing nut and a second Kirschner-wire fixing nut, wherein fixing said bone penetrating elements within said first and second recesses is accomplished by tightening said first and second Kirschner-wire fixing screws in said first and second Kirschner-wire fixing nuts, respectively.

14. The fixator as claimed in claim 12 wherein at least one of said lateral cages further comprises:
a first hole and a second hole drilled in the body of said lateral cages and sized to fit said connecting rod, wherein said first hole and said second hole are substantially perpendicular to each other, and wherein said first hole and second hole intersect;
a tapped hole drilled in the body of said lateral cages, substantially perpendicular to said first hole and said second hole, and intersecting said first hole and second hole; and
a screw, wherein tightening said screw within said tapped hole causes the body of said lateral cages to be firmly fixed to said connecting rod inserted in one of said first hole and second hole.

15. The fixator as claimed in claim 1, further comprising:
at least a second connecting rod, wherein each of said first rod and second rod comprises at least one sphere; and
at least one central cage joint capable of flexibly joining said first and second rods by interfacing with said spheres, and further capable of fixing the relative orientation of said rods.

16. The fixator as claimed in claim 1, further combined or interlocked with an internal fixation device, wherein said internal device is selected from a group of Kirschner wires, threaded Kirschner wires, pins, and screws.

17. A method of externally fixating fractured or osteotomized bones comprising:
providing at least two bone penetrating elements;
passing at least one of the bone penetrating elements through the bone;
providing at least a first connecting rod; and
providing at least two lateral cages, wherein each of the lateral cages has at least one recess through which the bone penetrating elements are held and fixed, wherein each of the lateral cages is capable of connecting said at least two bone penetrating elements to said at least first connecting rod.

18. The method as claimed in claim 17, wherein said at least two penetrating elements are preloaded after inserted through the bone in a linear drilling manner and then connected or interlocked into at least one recess of the lateral cage.

19. The method as claimed in claim 18, wherein at least one of said at least two penetrating elements is drilled in a non-linear manner.

20. The method as claimed in claim 17, wherein at least one of said at least two penetrating elements is drilled in a linear manner.
